(19) United States
(12) Patent Application Publication
(10) Pub. No.: US 2010/0114271 A1
(43) Pub. Date: May 6, 2010

(54) SHIELDED CONDUCTOR FILAR - STIMULATION LEADS

(75) Inventors: John L. Sommer, Coon Rapids, MN (US); Scott Brabec, Elk River, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/262,306

(22) Filed: Oct. 31, 2008

(51) Int. Cl. H01B 3/44; A61N 1/04

(52) U.S. Cl. 607/115; 174/110 R

(57) ABSTRACT

A lead for a medical device includes an elongate filar core member having an axis, which is operable for transmitting a lead signal. Furthermore, the lead includes an insulating layer disposed directly on the elongate filar core member and that extends along the axis. The lead also includes an electrically conductive layer disposed directly on the insulating layer and that extends along the axis.

### FIELD

The present disclosure relates generally to medical devices and, more specifically, to a shielded conductor filar for a stimulation lead.

### BACKGROUND

The statements in this section merely provide background information related to the present disclosure and may not constitute prior art.

Various elongate, conductive leads have been proposed for transmitting a signal for a medical device. For example, conductive leads have been proposed for functioning as a heart pacemaker lead, as a defibrillation lead, as a neural lead, and the like. For example, in the case of the pacemaker lead, the lead transmits a pacing signal from a pacemaker device to corresponding heart tissue to maintain proper heart function.

Conventional leads, typically include a single conductive wire (i.e., filar) or coil with a protective coating thereon. These conventional leads typically function within an independent circuit. As such, the usefulness of these leads may be somewhat limited. Furthermore, these leads may be prone to fracture, which can prevent proper signal transmission. Additionally, an electromagnetic field can leak into the lead and add noise to the signal. For example, a patient with an implanted pacemaker lead may not be able to undergo an MRI imaging procedure because the resultant electromagnetic field may detrimentally effect operation of the signal transmission within the lead.

### SUMMARY

This section provides a general summary of the disclosure, and is not a comprehensive disclosure of its full scope or all of its features.

A lead for a medical device is disclosed. The lead includes an elongate filar core member having an axis, which is operable for transmitting a lead signal. Furthermore, the lead includes an insulating layer disposed directly on the elongate filar core member and that extends along the axis. The lead also includes an electrically conductive layer disposed directly on the insulating layer and that extends along the axis.

In addition, a method of manufacturing a lead for a medical device is disclosed. The method includes coating an elongate filar core member with an insulating layer, wherein the elongate filar core member is operable for transmitting a lead signal. The method also includes depositing an electrically conductive layer directly on the insulating layer.

Moreover, a method of operating a medical device is disclosed. The method includes operatively connecting a medical lead in a predetermined anatomical location. The medical lead includes an elongate filar core member having an axis, an insulating layer disposed directly on the elongate filar core member and extending along the axis, and an electrically conductive layer disposed directly on the insulating layer and extending along the axis. Furthermore, the method includes transmitting a lead signal via the elongate filar core member.

Additionally, a lead for a medical device is disclosed that includes an elongate filar core member. The elongate filar core member is operable for transmitting a lead signal. The elongate filar core member has an axis, a cross section of the elongate filar core member substantially perpendicular to the axis is substantially solid, and the elongate filar core member is made of silver, MP35N, MP35N-clad silver, platinum, platinum clad tantalum, silica, or a combination thereof. Also, the lead includes an insulating layer disposed directly on the elongate filar core member and extends along the axis. The insulating layer is made of soluble imide (SI) polyimide or Ethylene Tetrafluoroethylene (ETFE) insulating polymer. The insulating layer substantially surrounds an outer surface of the elongate filar core member. The lead also includes an electrically conductive layer disposed directly on the insulating layer and extends along the axis. The electrically conductive layer is made of gold, platinum, carbon, carbon nanotubes, or a combination thereof. The electrically conductive layer is operable for transmitting the lead signal, transmitting a secondary signal that is different from the lead signal, and/or providing shielding for transmission of the lead signal. Also, the electrically conductive layer substantially surrounds an outer surface of the insulating layer. Still further, the lead includes a protective layer that substantially surrounds an outer surface of the electrically conductive layer.

Further areas of applicability will become apparent from the description provided herein. The description and specific examples in this summary are intended for purposes of illustration only and are not intended to limit the scope of the present disclosure.

### DRAWINGS

The drawings described herein are for illustration purposes only and are not intended to limit the scope of the present disclosure in any way.

FIG. 1 is a schematic view of a medical device with a lead according to the teachings of the present disclosure;

FIG. 2 is a perspective view of the lead of FIG. 1 shown partially exposed;

FIG. 3 is a sectional view of the lead of FIG. 1;

FIG. 4 is a sectional view of a lead according to another embodiment;

FIG. 5 is a sectional view of a lead according to still another embodiment;

FIG. 6 is a longitudinal sectional view of the lead of FIG. 1;

FIG. 7 is a schematic electrical diagram that includes the lead of FIG. 1 according to an exemplary embodiment; and

FIG. 8 is a schematic electrical diagram that includes the lead of FIG. 1 according to another exemplary embodiment.

### DETAILED DESCRIPTION

The following description is merely exemplary in nature and is not intended to limit the present disclosure, application, or uses. It should be understood that throughout the drawings, corresponding reference numerals indicate like or corresponding parts and features.

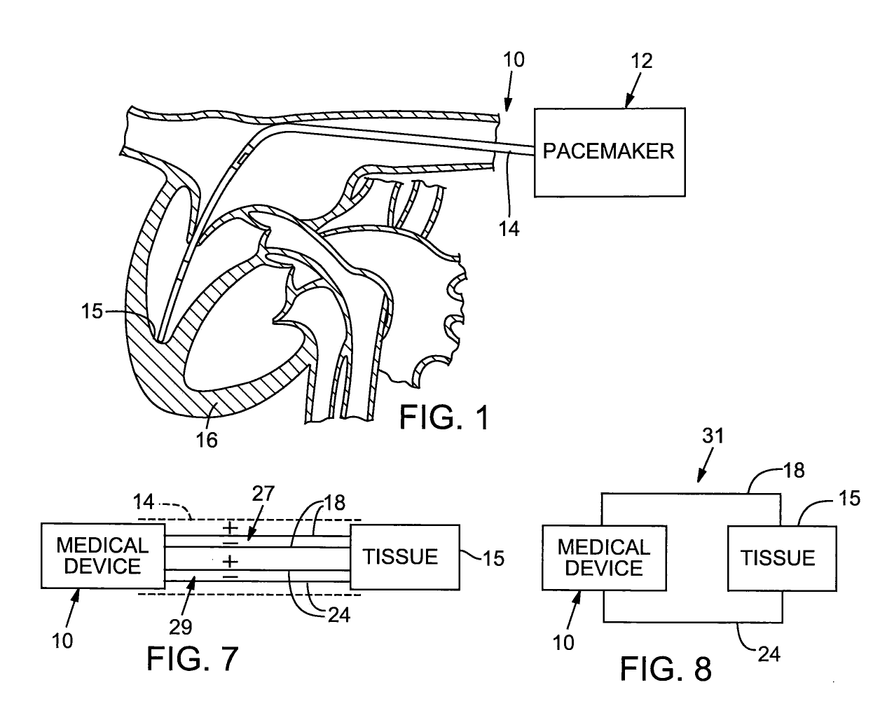

Referring initially to FIG. 1, an exemplary embodiment of a medical device 10 is schematically illustrated. In this embodiment, the medical device is a pacemaker device 12 or other cardiac rhythm management device. The pacemaker device 12 can be of a known type.

A lead 14 is operatively connected to the medical device 10. The lead 14 is generally elongate and extends between the pacemaker device 12 and biological tissue 15, such as tissue of a heart 16, at a predetermined location. Thus, the lead 14 is operatively coupled to the pacemaker device 12 and the tissue 15 to transmit a lead signal (e.g., a stimulation signal or sensing signal to/from the heart).

It will be appreciated that the medical device 10 and the signals transmitted by the lead 14 could be of any suitable type without departing from the scope of the present disclosure. For instance, the lead 14 could stimulate and/or sense neural signals to/from brain tissue, the lead 14 could transmit signals for heart defibrillation, or any other type. Furthermore, the lead 14 could be an implantable lead for longer term use, or the lead 14 could be temporarily coupled to tissue 15 without departing from the scope of the present disclosure.

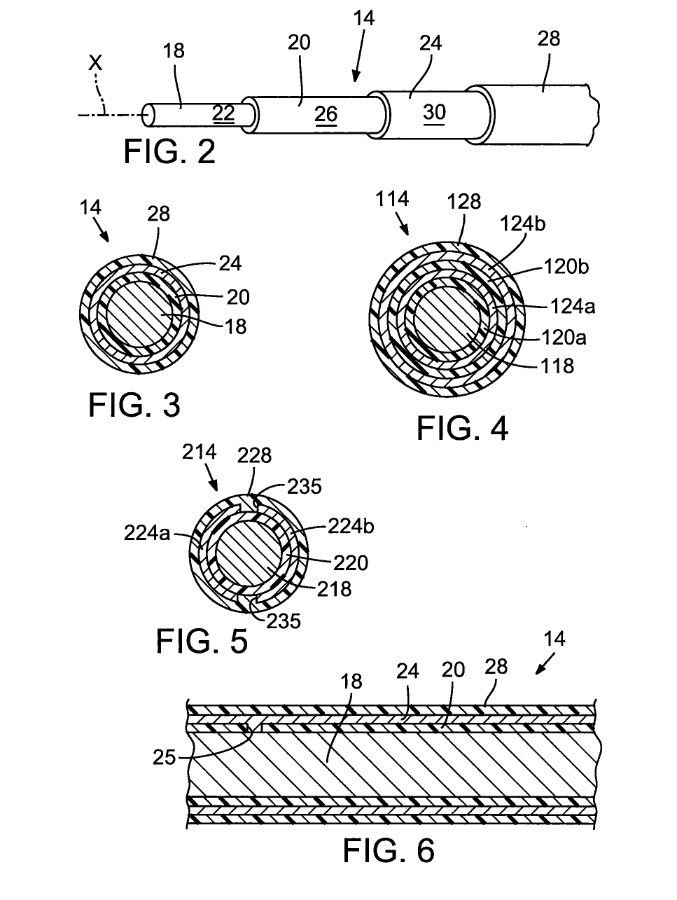

FIGS. 2 and 3 illustrate an exemplary embodiment of the lead 14 of FIG. 1 in greater detail. It will be appreciated that FIG. 2 shows the lead 14 with certain outer layers removed for clarity; however, these layers could extend over the entire axial length of the lead 14 without departing from the scope of the present disclosure.

As shown, the lead 14 includes an elongate filar (i.e., thread-like) core member 18. The filar core member 18 defines an axis X. In some embodiments, the filar core member 18 is flexible. Also, in some embodiments, the filar core member 18 has a cross section that is substantially solid. For instance, the filar core member 18 can have a diameter of approximately 0.004 inches. Furthermore, in some embodiments, the filar core member 18 can include a lumen that extends along the axis X.

The filar core member 18 can be made out of any suitable material. For instance, in some embodiments, the filar core member 18 is made out of an electrically conductive material. Also, in some embodiments, the filar core member 18 is made out of an optical fiber for transmitting an optic signal (i.e., light). Accordingly, the filar core member 18 can be made out of silver, MP35N stainless steel, MP35N-clad silver, platinum, platinum-clad tantalum, silica, or a combination of two or more of these materials. However, it will be appreciated that the filar core member 18 could be made out of any suitable material without departing from the scope of the present disclosure. Furthermore, in cases in which the filar core member 18 is made out of an optical fiber, the filar core member 18 can be coated with a reflective material to enhance the transmission of optical signals (i.e., light) therethrough.

In addition, in the embodiments represented in FIGS. 2 and 3, the lead 14 includes an insulating layer 20. In some embodiments, the insulating layer 20 is generally hollow and cylindrical and is disposed directly on an outer surface 22 of the core member 18 to cover and surround the outer surface 22. The insulating layer 20 can extend over the majority of the axial length of the filar core member 18. Also, in some embodiments, the insulating layer 20 can leave a portion of the outer surface 22 exposed for operative and electrical connection of the filar core member 18 to the medical device 10 and/or the tissue 15.

The insulating layer 20 can have any suitable thickness. For example, in some embodiments, the insulating layer 20 has a substantially constant wall thickness of approximately 0.0002 inches.

In some embodiments, the insulating layer 20 is made out of an electrically insulating material, such as an insulating polymeric material. For example, in some embodiments, the insulating layer 20 can be formed of polyimide, such as a Soluble Imide (SI) polyimide material as described in U.S. Pat. No. 5,639,850, issued to Bryant on Jun. 17, 1997, and incorporated herein by reference in its entirety. In other embodiments, the insulating layer 20 is made out of Ethylene Tetrafluoroethylene (ETFE) insulating polymer. As such, the insulating layer 20 can effectively insulate and protect the filar core member 18. Also, manufacturing of the lead 14 can be facilitated due to the insulating layer 20 as will be discussed.

Moreover, the lead 14 can also include an electrically conductive layer 24. In some embodiments, the conductive layer 24 is generally hollow and cylindrical and is disposed directly on an outer surface 26 of the insulating layer 20 to cover and surround the outer surface 26. As such, the conductive layer 24 is supported by the filar core member 18, and the insulating layer 20 is disposed between the filar core member 18 and the conductive layer 24. The conductive layer 24 can extend over the majority of the axial length of the insulating layer 20. Also, in some embodiments, the insulating layer 20 can leave a portion of the outer surface 22 of the filar core member 18 exposed for operative and electrical connection of the filar core member 18 to the medical device 10 and/or the tissue 15.

The conductive layer 24 can have any suitable thickness. For example, in some embodiments, the conductive layer 24 has a substantially constant wall thickness of approximately 0.0002 inches.

The conductive layer 24 can be made out of any suitable material, such as an electrically conductive material. For example, in some embodiments, the conductive layer 24 can be formed of gold, platinum, carbon, carbon nanotubes, or a combination of two or more of these materials.

As will be discussed in greater detail below, the conductive layer 24 can be operable for transmitting the same signal (i.e., the lead signal) as the filar core member 18, can transmit a separate signal (i.e., a secondary signal) from the filar core member 18, or can provide shielding of the core member 18 from signal leakage for improved transmission of the lead signal by the core member 18. Furthermore, manufacturing of the lead 14 can be facilitated due to the conductive layer 24 as will be discussed.

Additionally, in the embodiments represented in FIGS. 2 and 3, the lead 14 includes a protective layer 28. In some embodiments, the protective layer 28 is generally cylindrical and hollow and is disposed directly on an outer surface 30 of the conductive layer 24 to cover and surround the outer surface 30. The protective layer 28 can extend over the majority of the axial length of the lead 14. Also, in some embodiments, the protective layer 28 can leave a portion of the outer surface 22 of the filar core member 18 and/or the outer surface 30 of the conductive layer 24 exposed for operative and electrical connection to the medical device 10 and/or the tissue 15.

The protective layer 28 can have any suitable thickness. For example, in some embodiments, the protective layer 28 has a substantially constant wall thickness of approximately 0.0002 inches.

The protective layer 28 can be made out of any suitable material, such as an electrically insulative material. For example, in some embodiments, the protective layer 28 can be made out of Si polyimide, similar to the insulating layer 20. In other embodiments, the protective layer 28 is made out of ETFE insulating polymer similar to the insulating layer 20. Accordingly, the protective layer 28 can protect the other components of the lead 14 from abrasion or other damage. Furthermore, the protective layer 28 can facilitate manufacturing of the lead 14 as will be discussed.

To manufacture the lead 14, in some embodiments, the insulating layer 20 is first coated on the filar core member 18. For example, in some embodiments, the material of insulating layer 20 is combined with a solvent, such as Naptha or petroleum ether, into a liquid, and the filar core member 18 is exposed to the solvent-based combination. Then, heat (e.g., 650° to 750° F.) is applied to drive out the solvent, thereby curing the insulating layer 20. In some embodiments, this process is repeated multiple times in order to build up the insulating layer 20 in layers until insulating layer 20 has the desired wall thickness. For instance, the core member 18 can exposed to the solvent-based combination and cured between fifteen times and twenty times, and in some embodiments, a total of eighteen times. However, it will be appreciated that the insulating layer 20 can be formed in any suitable fashion.

After the insulating layer 20 has cured, material of the conductive layer 24 can be deposited thereon. In some embodiments, the conductive layer 24 is formed by a known sputtering process, in which the insulating layer 20 is bombarded by atomized particles of the material of the conductive layer 24. In other embodiments, such as where the conductive layer 24 is made out of carbon nanotubes, the carbon nanotubes are mixed with polyimide in a liquid state, and the lead 14 is dipped into the liquid mixture to deposit the mixture on the insulating layer 20. However, it will be appreciated that the conductive layer 24 can be formed in any suitable fashion.

Next, the protective layer 28 is formed on the conductive layer 24. In some embodiments, the protective layer 28 is formed in a manner that is substantially similar to that of the insulating layer 20.

The lead 14 can be operatively connected to the medical device 10 and/or the tissue 15 using any suitable fastener. Also, the core member 18 and the conductive layer 24 of the lead 14 can be electrically connected to the medical device 10 and/or tissue 15 in order to create one or more circuits therewith. For instance, the core member 18 and/or the conductive layer 24 can include specific electrodes (not shown) (e.g., exposed areas) for operatively connecting with the medical device 10 and/or the tissue 15. Also, in some embodiments represented in FIG. 6, an aperture 25 can be formed in the insulating layer 20 such that the core member 18 and conductive layers 24 abut so as to electrically connect together.

Thus, as represented in FIG. 7, the core member 18 can be incorporated into a first circuit 27 that transmits a lead signal, and the conductive layer 24 can be incorporated into a separate, independent second circuit 29 for transmitting a secondary signal. Also, as represented in FIG. 8, the core member 18 and conductive layers 24 can be connected in a circuit 31 with the medical device 10 and the tissue 15 such that the core member 18 operates as a cathode within the circuit, and the conductive layers 24 operates as an anode within the circuit, or vice versa. Furthermore, the conductive layer 24 can be electrically connected to the core member 18 and operate to redundantly transmit the same signal as the core member 18. Similarly, the conductive layer 24 can operate as a shunt to the core member 18 in the event that the core member 18 fractures, builds up excessive resistance, and the like.

Moreover, in some embodiments, the conductive layer 24 can shield the core member 18 from signal leakage. For instance, in some embodiments, the conductive layer 24 is electrically connected to ground, and the core member 18 transmits the lead signal. Because the conductive layer 24 is substantially continuous (i.e., does not include any substantial gaps), because the conductive layer 24 covers substantially the entire axial length of the core member 18, and because the conductive layer 24 is spaced from the core member 18 by the thickness of the insulating layer 20, the conductive layer 24 can substantially reduce leakage of the signal into and/or out of the core member 18. Accordingly, the signal is less likely to be detrimentally effected by signal noise and/or the signal is more likely to transmit at a sufficient strength. In some embodiments, the conductive layer 24 shields the core member 18 against electromagnetic fields due to MRI imaging procedures. Also, in some embodiments, the lead 14 can be operatively connected to a sensor (not shown) for transmitting signals to and/or from the tissue 15, and the conductive layer 24 shields the core member 18 from signal leakage for more accurate operation of the sensor.

Thus, the lead 14 can be used in a wide variety of ways and for a wide variety of functions. Because of the conductive layer 24, the lead 14 can be more versatile for transmitting a wider variety of signals. Also, the conductive layer 24 can enable the lead 14 to transmit signals even if the core member 18 fails. Moreover, the conductive layer 24 can provide shielding for improving signal transmission. Additionally, the lead 14 can be manufactured relatively quickly and in a relatively inexpensive manner as compared to some conventional leads.

Referring now to FIG. 4, another embodiment of the lead 114 is illustrated. Components that are similar to those of FIGS. 1-3 are identified with corresponding reference numerals increased by 100.

As shown, the lead 114 includes a core member 118, a first insulating layer 120a, and a first conductive layer 124a similar to the embodiment of FIG. 3. However, the lead 114 additionally includes a second insulating layer 120b disposed over and covering the first conductive layer 124a and a second conductive layer 124b disposed over and covering the second conductive layer 124b. Also, the lead 114 includes a protective layer 128 disposed over and substantially covering the second conductive layer 124b.

It will be appreciated that the core member 118 and the first and second conductive layers 124a, 124b can each be operatively connected to the medical device 10 and/or the tissue 15 for signal transmission as discussed above. Also, it will be appreciated that the first and/or second conductive layers 124a, 124b can provide shielding for the signal transmission within the core member 118. Moreover, the lead 114 can include any number of conductive layers 124a, 124b for increasing the versatility of the lead 114 and/or for increasing the shielding capability of the lead 114.

Referring now to FIG. 5, another embodiment of the lead 214 is illustrated. Components that are similar to those of FIGS. 1-3 are identified with corresponding reference numerals increased by 200.

As shown, the lead 214 includes a core member 218 and an insulating layer 220 similar to the embodiment of FIGS. 1-3. The lead 214 also includes a first conductive layer 224a and a second conductive layer 224b. The first conductive layer 224a is a layer of conductive material that extends along the axis of the lead 214 and, as shown in the cross section of FIG. 5, the first conductive layer 224a covers only a portion of the core member 218. Similarly, the second conductive layer 224b is a layer of conductive material that extends along the axis of the lead 214 and, in cross section, the second conductive layer 224b covers only a portion of the core member 218. In some embodiments, the first and second conductive layers 224a, 224b are disposed in spaced relationship on opposite sides of the core member 218 so as to be substantially symmetrically disposed about the axis X. Thus, gaps 235 are defined between the first and second conductive layers 224a, 224b as shown. In some embodiments, the first conductive layer 224a covers approximately one hundred and seventy degrees about the circumference of the core member 218 and the second conductive layer 224b extends approximately one hundred and seventy degrees about the circumference of the core member 218, leaving gaps 235 totaling approximately twenty degrees.

Furthermore, the lead 214 can include a protective layer 228. The protective layer 228 encapsulates and surrounds the other components of the lead 214 and fills the gaps 235 between the first and second conductive layers 224a, 224b.

It will be appreciated that the lead 214 could include any number of conductive layers 224a, 224b without departing from the scope of the present disclosure. It will also be appreciated that the core member 218 and the conductive layers 224a, 224b could be operatively coupled to the medical device 10 and tissue 15 for signal transmission as discussed above.

Certain terminology is used herein for purposes of reference only, and thus is not intended to be limiting. For example, terms such as “upper,” “lower,” “above,” “below,” “top,” “upward,” and “downward” refer to directions in the drawings to which reference is made. Terms such as “front,” “back,” “rear,” and “side,” describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first,” “second,” and other such numerical terms referring to structures do not imply a sequence or order unless clearly indicated by the context.

When introducing elements or features and the exemplary embodiments, the articles “a,” “an,” “the” and “said” are intended to mean that there are one or more of such elements or features. The terms “comprising,” “including,” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

The description of the disclosure is merely exemplary in nature and, thus, variations that do not depart from the gist of the disclosure are intended to be within the scope of the disclosure. Such variations are not to be regarded as a departure from the spirit and scope of the disclosure.

What is claimed is:

1. A lead for a medical device comprising:
an elongate filar core member having an axis, the elongate filar core member operable for transmitting a lead signal;
an insulating layer disposed directly on the elongate filar core member and extending along the axis; and
an electrically conductive layer disposed directly on the insulating layer and extending along the axis.

2. The lead of claim 1, wherein the insulating layer substantially surrounds an outer surface of the elongate filar core member, and the electrically conductive layer substantially surrounds an outer surface of the insulating layer.

3. The lead of claim 1, further comprising a protective layer that substantially surrounds an outer surface of the electrically conductive layer.

4. The lead of claim 1, wherein a cross section approximately perpendicular to the axis of the elongate filar core member is substantially solid.

5. The lead of claim 1, wherein the elongate filar core member is made of at least one of an electrically conductive material and an optical fiber material.

6. The lead of claim 5, wherein the elongate filar core member includes a material chosen from a group consisting of silver, MP35N, MP35N-clad silver, platinum, platinum clad tantalum, silica, and a combination thereof.

7. The lead of claim 1, wherein the insulating layer includes a polymeric material.

8. The lead of claim 7, wherein the polymeric material is made out of at least one of soluble imide (SI) polyimide and Ethylene Tetrafluoroethylene (ETFE) insulating polymer.

9. The lead of claim 1, wherein the electrically conductive layer includes a material chosen from a group consisting of gold, platinum, carbon, carbon nanotubes, and a combination thereof.

10. The lead of claim 1, wherein the electrically conductive layer is operable for at least one of transmitting the lead signal, transmitting a secondary signal that is different from the lead signal, and providing shielding for transmission of the lead signal.

11. The lead of claim 1, further comprising a first electrically conductive layer, a second electrically conductive layer, and a second insulating layer, the second electrically conductive layer supported by the first electrically conductive layer, and the second insulating layer disposed between the first and second electrically conductive layers.

12. The lead of claim 1, wherein in a cross section substantially perpendicular to the axis, the electrically conductive layer covers only a portion of the elongate filar core member.

13. A method of manufacturing a lead for a medical device comprising:
coating an elongate filar core member with an insulating layer, the elongate filar core member operable for transmitting a lead signal; and
depositing an electrically conductive layer directly on the insulating layer.

14. The method of claim 13, further comprising coating the electrically conductive layer with a protective layer.

15. The method of claim 13, wherein coating the elongate filar core member comprises exposing the elongate filar core member to the insulating material, which is in a liquid state.

16. The method of claim 13, wherein depositing the electrically conductive layer comprises sputtering the electrically conductive layer onto the insulating layer.

17. A method of operating a medical device comprising:
operatively connecting a medical lead in a predetermined anatomical location, the medical lead including an elongate filar core member having an axis, an insulating layer disposed directly on the elongate filar core member and extending along the axis, and an electrically conductive layer disposed directly on the insulating layer and extending along the axis; and
transmitting a lead signal via the elongate filar core member.

18. The method of claim 17, further comprising at least one of transmitting the lead signal via the electrically conductive layer, transmitting a secondary signal that is different from the lead signal via the electrically conductive layer, and shielding transmission of the lead signal.

19. A lead for a medical device comprising:
an elongate filar core member, the elongate filar core member operable for transmitting a lead signal, wherein the elongate filar core member has an axis, and wherein a cross section of the elongate filar core member substantially perpendicular to the axis is substantially solid, wherein the elongate filar core member includes a material chosen from a group consisting of silver, MP35N, MP35N-clad silver, platinum, platinum clad tantalum, silica, and a combination thereof;
an insulating layer disposed directly on the elongate filar core member and extending along the axis, wherein the insulating layer is made out of at least one of soluble imide (SI) polyimide and Ethylene Tetrafluoroethylene (ETFE) insulating polymer, wherein the insulating layer substantially surrounds an outer surface of the elongate filar core member;
an electrically conductive layer disposed directly on the insulating layer and extending along the axis to substantially surround an outer surface of the insulating layer, wherein the electrically conductive layer includes a material chosen from a group consisting of gold, platinum, carbon, carbon nanotubes, and a combination thereof, wherein the electrically conductive layer is operable for at least one of transmitting the lead signal, transmitting a secondary signal that is different from the lead signal, and providing shielding for transmission of the lead signal; and
a protective layer that substantially surrounds an outer surface of the electrically conductive layer.
